(19) United States
(12) Patent Application Publication
(10) Pub. No.: US 2014/0125532 A1
(43) Pub. Date: May 8, 2014

(54) TATTOOED ANTENNAS

(71) Applicant: University of Utah, Salt Lake City, UT (US)

(72) Inventors: Cynthia Furse, Salt Lake City, UT (US); Patrick A. Tresco, Sandy, UT (US)

(21) Appl. No.: 14/075,712

(22) Filed: Nov. 8, 2013

Related U.S. Application Data
(60) Provisional application No. 61/724,209, filed on Nov. 8, 2012.
(60) Provisional application No. 61/765,507, filed on Feb. 15, 2013.

(51) Int. Cl. H01Q 1/27

(52) U.S. Cl. CPC H01Q 1/273; B82Y 5/00
USPC 343/718; 977/773; 977/904; 977/950

(57) ABSTRACT

A tattooed antenna and antenna system are disclosed. The tattooed antenna includes one or more nanoparticles in a fluid. The one or more nanoparticles are configured to be injected as a tattoo into a body to thereby form an antenna configured to transmit data received from a source. The antenna system may include feed system that drives the tattooed antenna. The tattooed antenna may be a feed pickup antenna and/or a radiating antenna.

### CROSS-REFERENCE TO RELATED APPLICATIONS

This application is a non-provisional of, and claims priority to and the benefit of, U.S. Provisional Patent Application Ser. No. 61/724,209 filed on Nov. 8, 2012 and entitled “TATTOOED ANTENNAS,” and U.S. Provisional Patent Application Ser. No. 61/765,507 filed on Feb. 15, 2013 and entitled “TATTOOED ANTENNAS,” both of which applications are hereby expressly incorporated herein by this reference in their entirety.

### BACKGROUND

Implantable medical devices touch virtually every major function in the human body. Cardiac pacemakers and defibrillators, neural recording and stimulation devices, cochlear, and retinal, implants are just a few of the many implantable medical devices available today. Wireless telemetry for these devices is necessary to monitor battery level and device health, upload reprogramming for device function, and download data for patient monitoring. Antennas are inevitably one of the largest if not the largest component of the telemetry communication system and are generally mounted on or in the implanted battery pack, usually in a body cavity. This limited real estate significantly constrains the performance of implantable antennas. Typical battery packs for cardiac devices are under 4 cm long and new devices for neural recording and stimulation are less than 4 mm in size. Half wave antennas in the MedRadio band (402-405 MHz) are 36 cm in air and 6 cm plus in the body. In addition, implantation in the body cavity means the antennas have to transmit several cm through multiple layers of body tissue, where power may be lost. Lost power means lost distance and lost battery life.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 illustrates an environment and system for implementing a tattooed radiating antenna according to the embodiments disclosed herein;

FIG. 2 illustrates a simplified structure of a body layer and various locations where an antenna may be placed according to the embodiments disclosed herein;

FIG. 3 illustrates an example tattooed radiating antenna according to the embodiments disclosed herein;

FIG. 4 illustrates conductivity as a function of volume fraction of gold in a modeled tattooed radiating antenna according to the embodiments disclosed herein;

FIG. 5 illustrates a modeled tattooed radiating antenna according to the embodiments disclosed herein;

FIG. 6 illustrates a modeled tattooed radiating antenna according to the embodiments disclosed herein;

FIG. 7 illustrates an alternative environment for implementing an implantable antenna according to the embodiments disclosed herein;

FIGS. 8A-8C illustrate a modeled system including a feed antenna and a tattooed antenna according to the embodiments disclosed herein.

### DETAILED DESCRIPTION

Exemplary embodiments of the invention will become more fully apparent from the following detailed description and appended claims, taken in conjunction with the accompanying drawings. It is understood that this discussion describes only exemplary embodiments and are, therefore, not to be considered limiting of the invention's scope.

Embodiments disclosed herein are related to transforming the available size and efficiency of antennas for implantable medical devices and for other reasonable applications such as monitoring. The principles of the embodiments disclosed herein are a novel improvement over existing antenna systems in that rather than miniaturizing the antenna to fit on an implanted device, the embodiments disclosed herein place only the feed system on the implanted device, and use conductive materials tattooed in the skin and/or an adjacent fat layer to form the antenna as will be described in more detail to follow. This will increase the options for antenna size, shape, and configuration. It will also bring the antenna closer to the surface of the body, thus significantly reducing the transmitted loss and power absorption in the deeper body tissues.

### Example Tattooed Antenna System

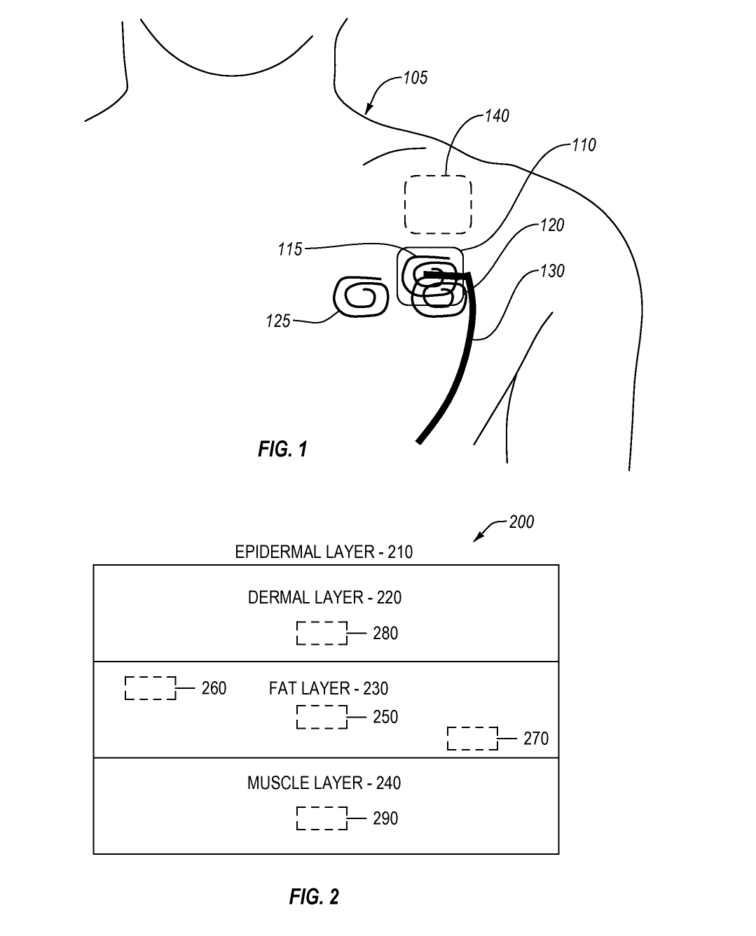

Attention is given to FIG. 1, which illustrates a system 100 that may be used to implement the embodiments disclosed herein. As illustrated, the system 100 may be implemented in a portion of a body 105. In the illustrated embodiment, the portion of a body 105 includes the chest cavity including the heart. It will be appreciated, however, that the portion of the body 105 may include any other portion of the body as needed. It will also be appreciated that the portion of the body 105 need not be a portion of a human body, but may be a portion of an animal body or another living organism as circumstances warrant.

The system 100 may include an implantable device 110. The implantable device may include a battery pack to power the device and may include other electronics that help the implantable device perform its intended function. Examples of implantable devices include a pacemaker as in the illustrated embodiment, deep brain stimulating electrodes, cochlear and other audio implants, and optical implants. It will be appreciated that the implantable device 110 may be any reasonable implantable device now known or that may be developed in the future. It will also be appreciated that the implantable device 110 may be implemented as a single device or may be implemented as a distributed device.

As also illustrated, the implantable device 110 includes a feed 115. The feed 115 is typically mounted directly to the implantable device 110 or a part of the device such as a battery pack in those embodiments where the battery pack is separate from the implantable device 110. The feed 115 may transmit signals to a subcutaneous layer of the body for pick-up by other elements of system 100 as will be explained in more detail to follow.

In some embodiments, the feed 115 may be or include a slot antenna or a microstrip feed antenna. In other embodiments, the feed 115 may be or include a rectangular patch, a spiral or serpentine microstrip, a bent dipole, an electrically small dipole, one or more inductive coils, or a genetic algorithm (GA) design. In some embodiments, the implantable device serves as the ground plane the feed 115. It will be appreciated that the principles of the embodiments disclosed herein are not limited to the type or design of the feed 115 as any reasonable implementation of the feed 115 is contemplated by the present disclosure.

The system 100 is also illustrated as including a tattooed feed pickup antenna 120. In operation, the tattooed feed pick-up antenna is operable to receive the signals provided by the feed 115. In some embodiments, the tattooed feed pickup antenna 120 is a passive feed pickup antenna in that the there is no physical or direct connection between the feed 115 and the antenna 120. Such passive coupling advantageously allows for tattooed pick-up antenna and a tattoo radiating antenna 130 to be placed near the surface of the skin as will be explained in more detail to follow. Of course, it will be appreciated that in some embodiments the feed 115 and the tattooed feed pickup antenna 120 may be directly connected if circumstances warrant such implementation. In some embodiments, the tattooed feed pickup antenna 120 may be a slot spiral antenna, a microstrip spiral antenna, a slot or aperture type antenna, or an inductive type antenna. It will be appreciated by one of skill in the art that the tattooed feed pickup antenna 120 may be any reasonable antenna and is not limited to any particular type of antenna.

The system 100 further includes a tattooed radiating antenna 130 that is coupled to the feed pickup antenna 120. As described, the tattooed radiating antenna 130 is located near the surface of the skin as will be explained in more detail. The tattooed feed pickup antenna 120 and/or the tattooed radiating antenna 130 may be made up of one or more conductive nanoparticles in a liquid that are tattooed near the surface of the skin. Any suitable nanoparticles may be used for implementing the tattooed feed pickup antenna 120 and/or the tattooed radiating antenna 130. For example, gold is used in yellow pigments; magnetite (FeO.Fe2O3) and charcoal (C) are often found in black tattoo ink; hematite (Fe2O3) and cinnabar (HgS) are used in red ink; limonite (FeO.OH.nH2O) is used for yellow pigment; corundum (Al2O3), rutile (TiO2), and zincite (ZnO) are used for white pigment, and blue pigment can be achieved with ferric ferrocyanide (Fe4[Fe(CN)6]3) and cobaltous aluminate (CoAl2O4). Synthetic organic pigments that are highly conjugated are also used and can be compounded with other elements. It will be appreciated that other suitable nanoparticles may also be used when practicing the principles of the present invention disclosed herein.

In operation, the tattooed radiating antenna 130 is driven by the tattooed feed pickup antenna 120. The tattooed radiating antenna 130 transmits the information provided by the feed 115 to an external signal processing device, where it may be analyzed as needed.

In some embodiments, the tattooed radiating antenna 130 may also receive data or other information from an external source. This data may then be provided to the implantable device 110 via the antennas 120 and 115. In one embodiment, this data may be used to charge the batteries of the implantable device 110. In other embodiments, this data may be used for other purposes as needed.

It will be appreciated that the tattooed radiating antenna 130 may be implemented as any reasonable antenna such as those previously discussed in relation to tattooed feed pickup antenna 120 or other antennas known in the art. In one embodiment, the tattooed radiating antenna 130 may be approximately 0.25 wavelengths long, although the principles of the current invention are not limited to such length and other lengths are contemplated.

As discussed, the tattooed feed pickup antenna 120 and/or the tattooed radiating antenna 130 may be tattooed near the surface of the skin. In one embodiment, tattooing is a process of intradermal injection that employs oscillating hollow needles that are inserted approximately 1.5 mm perpendicularly into the skin, and which inject approximately 100-200 μl of a nano-particulate formulation into the dermal skin layers. The present technology enables the controlled deposition of pigmented particulate formulations in discrete locations in the deep dermis. It will be appreciated that other known tattooing methods and procedures may also be used in practicing the embodiments disclosed herein as needed. Tattooing the antennas 120 and/or 130 allows for the antenna to be implemented at the surface of the skin, thus reducing the body transmission loss normally seen with conventional implantable antennas. Accordingly the antenna size is not constrained by the implantable device 110 size, shape or location.

As will be appreciated by one of skill in the art, significant power is lost (deposited in) the body as an antenna transmits from the implantable device 110, through the deep body tissues, to the outside. Additional power is lost (reflected) back into the body at the surface. The power lost (absorbed) in in the body effectively limits the total power that can be transmitted and hence the distance the system can transmit. RF safety regulations for localized power are based on a specific absorption rate (SAR)

$$SAR ( W \text{/} kg ) = \frac{σ {\vert {\overline{E}}_{p} \vert}^{2}}{2 δ}$$

where σ is the electrical conductivity of the tissue (S/m), Ēₚ is the peak value of the electric field, and δ is the density of the tissue (kg/m³). Localized SAR is limited to 2 W/kg in a 10-g region or 1.6 W/kg for 1-g. A major advantage of placing the tattooed radiating antenna 130 near the body surface in accordance with the principles of the present invention is the reduction in lost power since less power will be deposited in the portion of the body 105 and/or reflected.

In one embodiment, the system 100 may include an intermediate feed antenna 125 that is implemented as a deep body tattoo in a portion of the body that is further away from the surface of the skin than where the tattooed feed pickup antenna 120 and/or the tattooed radiating antenna 130 may be tattooed. The intermediate feed antenna 125 would then operate as an intermediary between the feed 115 and the tattooed feed pickup antenna 120. In other embodiments there may be multiple deep body tattoo antennas as circumstances warrant.

Returning again to FIG. 1, the system 100 may also include one or more additional tattooed radiating antennas. In FIG. 1, the one or more additional tattooed radiating antennas are illustrated by the dashed box 140, which represents a second tattooed radiating antenna located on the reverse side of the body portion 105 from the tattooed radiating antenna 130. The one or more additional tattooed radiating antennas 140 may be any type of antenna as known in the art and may function with the tattooed radiating antenna 130 to form any type of antenna array or group of arrays as known in the art. It will be appreciated that the one or more additional tattooed radiating antennas 140 may be located at any reasonable location on the body 105 as circumstances warrant. In some embodiments, the one or more additional tattooed radiating antennas 140 may use the same feed network as the tattooed radiating antenna 130 while in other embodiments the one or more additional tattooed radiating antennas 140 may have separate feed networks.

In some embodiments, the tattooed radiating antenna 130 may be implemented as a sensing antenna that is configured to sense one or more physical properties. For example, in the embodiment of FIG. 1, the tattooed radiating antenna 130 may be configured to sense the temperature of the body portion 105 or it may be configured to sense the amount of blood flow in the body portion 105. It will be appreciated by those of skill in the art that the tattooed radiating antenna 130 may be implemented as any type of reasonable sensing antenna as circumstances may warrant. Accordingly, the principles of the present invention disclosed herein are not limited to any particular type of sensing antenna.

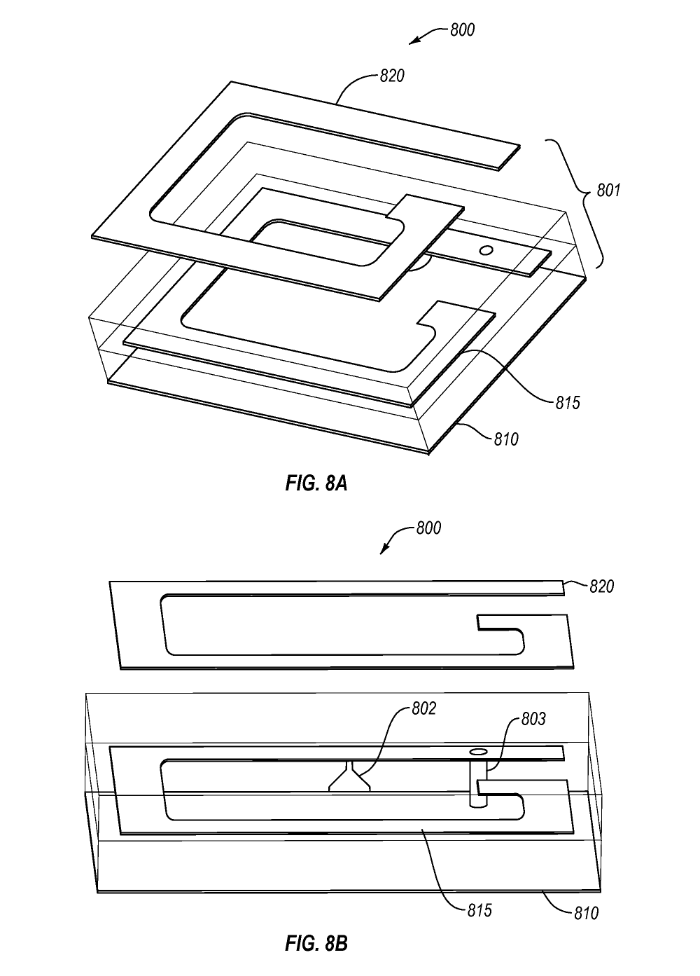

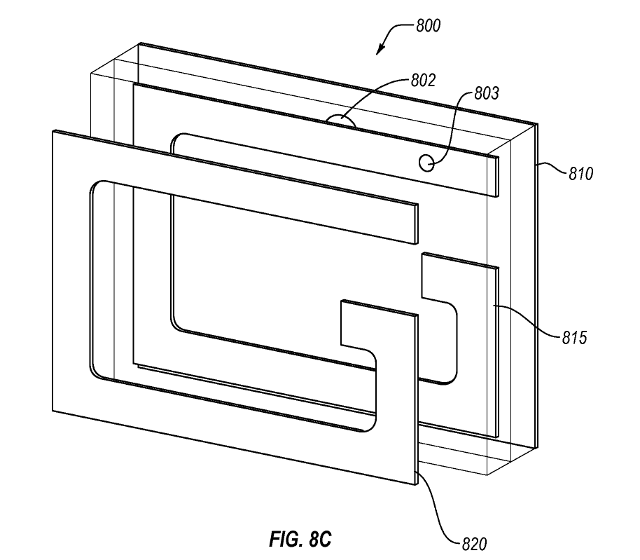

Attention is now given to FIGS. 8A-8C, which illustrate a modeled embodiment of a system 800 that includes a feed antenna and a tattooed antenna. As illustrated, the system 800, which may correspond to the system 100 previously discussed, includes an implantable device 810. It will be appreciated that the implantable device 810 may correspond to the implantable device 110 previously described and so need not be described in further detail.

As further illustrated, the system 800 includes a spiral feed antenna 815, which may be a specific embodiment of the feed antenna 115 previously described. As will be appreciated by one of skill in the art, the specific dimensions of the spiral feed antenna 815 are dependent on various operating parameters such as frequency. Accordingly, the spiral feed antenna 815 may have any reasonable dimensions to meet the specific operating parameters. It will also be appreciated that although the spiral feed antenna 815 is shown as being implemented as a single spiral antenna, in some embodiments one or more additional spiral antennas may be used to implement the spiral feed antenna 815.

The spiral feed antenna 815 may be coupled to the implantable device 810 in any reasonable way. As seen specifically in FIG. 8B, in the modeled embodiment the spiral feed antenna 815 may be coupled to the implantable device 810 via a feed connection 802 and a short connection 803. It will be appreciated by one of skill in the art that the feed connection 802 and a short connection 803 may be located at any reasonable location and may be implemented as any reasonable circuitry as circumstances warrant.

The FIGS. 8A-8C illustrate a gap or open space designated at 801. The gap 801 represents the layers of the body, such as body portion 105, that separate the spiral feed antenna 815 from the tattooed antenna 820. The layers of the body will be described in more detail to follow in relation to FIG. 2.

The FIGS. 8A-8C further illustrate a spiral tattooed antenna 820. The spiral tattooed antenna 820 may correspond to the feed pickup antenna 120, the tattooed radiating antenna 130, or a combination of both. The spiral tattooed antenna 820 in some embodiments may be located near the surface of the skin as previously described in relation to feed pickup antenna 120 and tattooed radiating antenna 130. In other embodiments, the spiral tattooed antenna 820 may be implemented in a piece of clothing that is worn near the surface of the skin. In operation, the spiral tattooed antenna 820 may couple through the body portion 801, either passively or directly, to the spiral feed antenna 815.

As will be appreciated by one of skill in the art, the specific dimensions of the spiral tattooed antenna 820 are dependent on various operating parameters such as frequency. Accordingly, the spiral tattooed antenna 820 may have any reasonable dimensions to meet the specific operating parameters. It will also be appreciated that although the spiral tattooed antenna 820 is shown as being implemented as a single spiral antenna, in some embodiments one or more additional spiral antennas may be used to implement the spiral tattooed antenna 820.

### Location of Tattooed Antenna

Attention is now given to FIG. 2, which illustrates a simplified structure 200 of the layers of the body 110 or the layers of the body represented by the gap 801. It will be appreciated that the structure 200 is not meant to be limiting and that other layers may also exist in the body 110 in addition to those illustrated. Further, it is also possible that layers that are not illustrated intervene between those that are illustrated. As illustrated, the body structure 200 includes an epidermal layer 210, which as is known is the top layer of the skin and a dermal layer 220 which is typically located directly beneath the epidermal layer 210. A fat layer 230 may be located beneath the dermal layer 220 and a muscle layer 240 may be located beneath the fat layer 240. As will be appreciated, the various layers illustrated may include water, blood, and other bodily fluids and materials. As is known, those layers located beneath the dermal layer are sometimes referred to collectively as the subcutaneous layer or layers. In one embodiment, the fat layer 230 may provide a relatively good insulator for the antennas 120 and 130, which may help to prevent short circuiting effects of the body. The muscle layer 240, which may be a moderate conductor, may provide a reasonable ground plane for the antennas 120 and 130. Table 1 shown below provides some electrical properties of these various layers as well as some other know materials.

TABLE 1
Electrical Properties of Tissues at 403.5 MHz

| | Relative | | | Skin | | |
|---|---|---|---|---|---|---|
| | Permittivity | Conductivity | Wavelength | depth ∫ | Density | |
| Tissue | εᵣ | σ (S/m) | λ_{ε} (cm) | (cm) | (kg/m³) | Thickness |
| Muscle | 57.95 | 0.82 | 9.3 | 5.2 | 1041 | 20 cm |
| ⅔ Muscle | 38.63 | 0.55 | 11.4 | 6.3 | ~1000 | |
| Fat | 11.62 | 0.081 | 14.9 | 22.6 | 916 | 0.8 cm |
| Skin (dry) | 46.71 | 0.68 | 10.4 | 5.6 | 1100 | Epidermis |
| Skin (wet) | 49.8 | 0.67 | 10.1 | 5.8 | 1100 | 62 μm |
| | | | | | | Dermis |
| | | | | | | 1676 μm |
| air | 1 | 0 | 74.3 | ∞ | 0 | |
| gold | | 4.567 × 10⁷ | | | | |

As mentioned, the antennas 120 and/or 130 may be tattooed in the portion of the body 105 so that the antennas are near the surface of the skin. As illustrated in FIG. 2, in one embodiment the antenna 120 and/or the antenna 130 may be tattooed so that they are centered in the fat layer 230. This is denoted by the reference 250.

In other embodiments, the antenna 120 and/or the antenna 130 may be located at the boundary of the dermal layer 120 and the fat layer 130 as denoted by reference 260. In an alternative embodiment, the antenna 120 and/or the antenna 130 may be located at the boundary of the fat layer 230 and the muscle layer 240 as denoted by reference 270. It will be appreciated that the antennas 120 and/or 130 may be centered at the boundaries or may be located at other locations at the boundaries as circumstances warrant. In still others embodiments, the antenna 120 and/or the antenna 130 may be located in the dermal layer 220 as denoted by reference 280. Alternatively, the antenna 120 and/or the antenna 130 may be located in the muscle layer 240 as denoted by reference 290.

It will be appreciated from the teachings of FIG. 2, that the antenna 120 and/or the antenna 130 may be located at any reasonable location near the epidermal layer 210. In addition, it will be appreciated that a deep body tattoo may be located in regions of the body that are further from the skin surface than shown in FIG. 2. Factors such as, but not limited to, required transmission frequency, size of the antennas, depth of the injection of the nanoparticles may help to determine the optimal location of the antenna 120 and/or the antenna 130 in the various layers of the structure 200. It will also be appreciated that the relative locations shown in FIG. 2 are for illustration only and are not meant to limit the principles of the present invention disclosed herein.

It will be appreciated that once the tattooed radiating antenna 130 is placed in a location, such as being centered in the fat layer 230, the thickness of the that layer, or any other layer where the tattooed radiating antenna 130 is located, may change due to weight loss or gain and other physical changes in the body. Accordingly, in the embodiments disclosed herein the tattooed radiating antenna 130 is configured to account for such changes so that the antenna is not detuned or otherwise degraded in its performance.

### Modeled Embodiment of a Tattooed Antenna

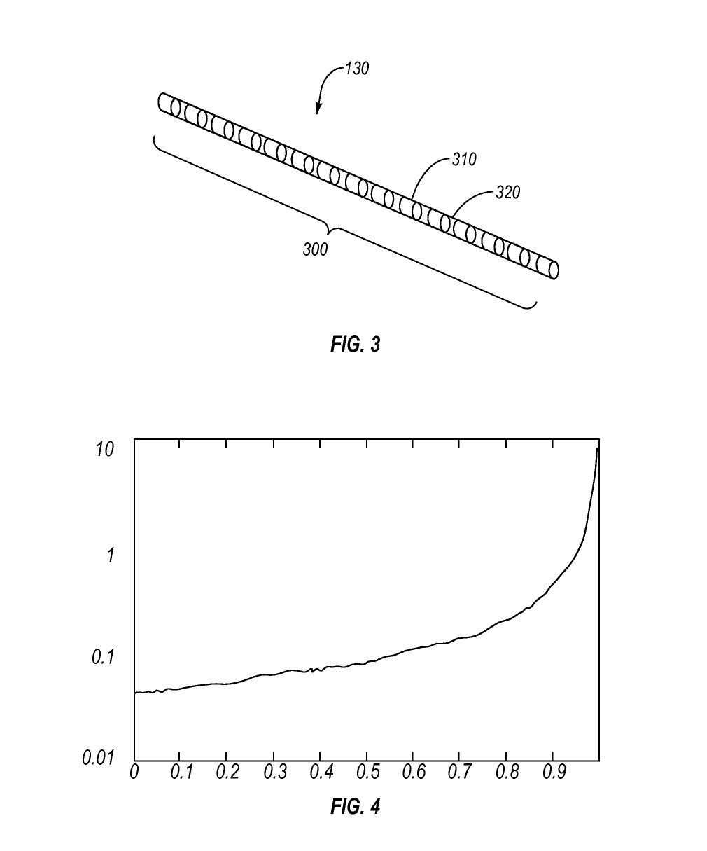

A specific modeled embodiment of the tattooed radiating antenna 130 will now be described. As shown in FIG. 3, the tattooed radiating antenna 130 in this embodiment is configured as a line implementing gold nanoparticles 300. Specifically, the antenna 130 was modeled as a set of collinear cylinders 310 separated by a cylindrical gap 320 filled with fat, as shown in FIG. 3. In the modeled embodiment, the tattooed radiating antenna 130 was implemented as a half wave tattooed dipole antenna using the gold nanoparticles 300 and was simulated at 402 MHz in a layered model of the body surface similar to that described above in relation to FIG. 2 having four layers; epidermis, dermis, fat, muscle, with the antenna centered in the fat layer.

In the modeled embodiment, the tattooed radiating antenna 130 was made from a 0.8 volume fraction line of gold cylinders 37.5 cm long as seen in FIG. 3. It will be appreciated that the length of the antenna 130 is based on a 0.25 (quarter) wavelength and that this may change depending on the frequency used, the depth of injection, and the matter surrounding the injection site.

FIG. 4 illustrates conductivity as a function of volume fraction of gold in the modeled tattooed radiating antenna 130. As shown, the conductivity increases approximately linearly up to 80% volume fraction.

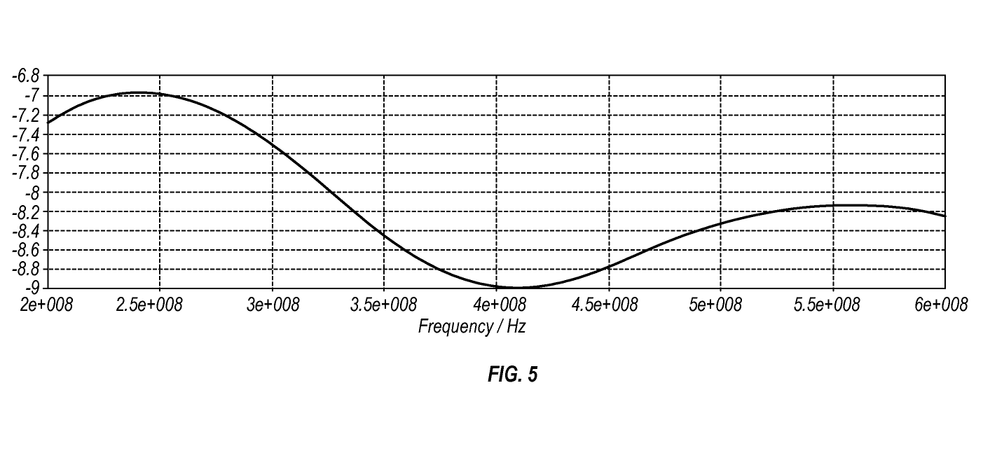

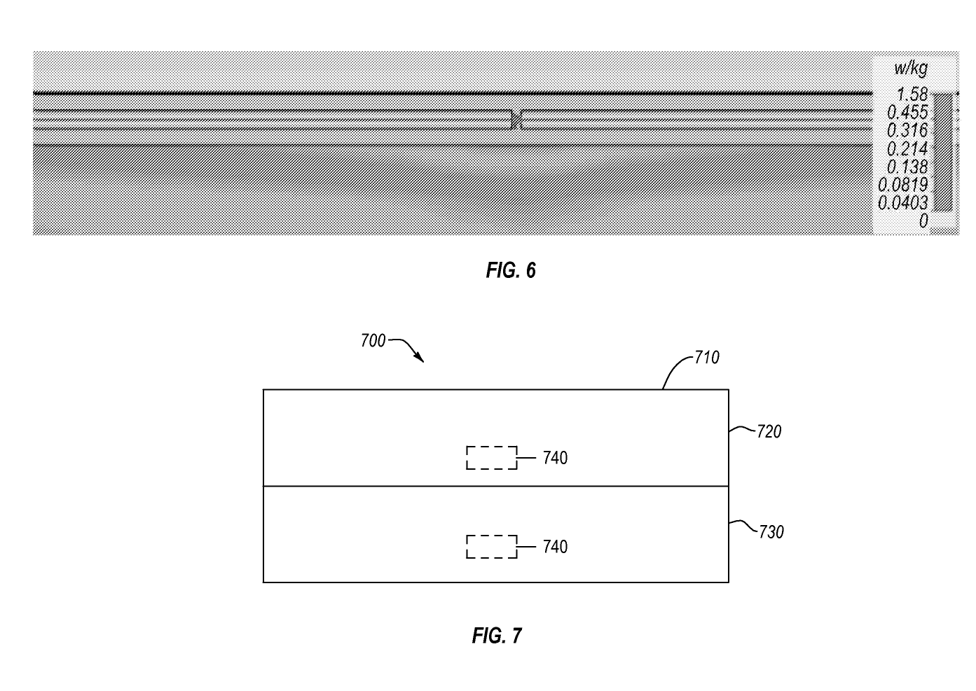

FIG. 5 illustrates that the modeled tattooed radiating antenna 130 has a low Q resonant dip of −9 dB at 402 MHz. It will be appreciated that this value is close to a dip of −10 dB that is generally desired. For a 1 g Specific Absorption Rate (SAR) limit of 1.6 W/kg (shown in FIG. 6), the total power that can be transmitted from modeled tattooed radiating antenna 130 is 27.6 mW, some of which may be lost in the body.

From FIG. 6 it can be seen that the SAR is clearly concentrated at the feed point, which may be expected from a simple dipole antenna. The SAR can be reduced, and hence the efficiency increased, by increasing the relative size of the feed (this will happen naturally for passive coupling with the feed and antenna being fed separated by some distance), isolating the feed from the body, perhaps by implanting a piece of silicone at the feed point location, or increasing the conductivity of the antenna (using more dense or thicker layers of nanoparticles). The modeled tattooed radiating antenna 130 has a realized power at the body surface of 6.3 mW. It will be appreciated that in most embodiments, the maximum transmit power of the tattooed radiating antenna 130 will be controlled by SAR regulations and limits.

It will be appreciated by one of skill in the art that the modeled tattooed radiating antenna 130 of FIG. 3 is only one of numerous embodiments that may be practiced using the principles of the present invention disclosed herein. That is, the principles of the current invention contemplate a tattooed radiating antenna 130 that uses different design parameters such as different nanoparticles, different operating frequencies, and different antenna lengths. Accordingly, the modeled tattooed radiating antenna 130 of FIG. 3 is to be viewed as illustrative only and not as limiting in any way the principles of the present invention disclosed herein.

### Flexible Electronics

In an alternative embodiment, the tattooed radiating antenna 130 may be implemented as a flexible electronics. A flexible electronic may include external, temporary stick-on plastic substrate that may be applied to the portion of the body 105 near the surface of the skin. The flexible electronics may also include other materials temporarily placed near the surface of the skin. In some embodiments, these devices are built with less than perfect conductive materials including printed and fluidic conductors. In such embodiments, the antenna 130 implemented as a flexible electronics resides near the surface of the skin and may thus be driven by the feed 115 in the manner previously described. In addition, the flexible electronics may take the form of any reasonable antenna as circumstances warrant. In some embodiments, the flexible electronics may be implemented as a textile antenna as is known in the art.

### Alternative Embodiments

Attention is now given to given to FIG. 7, which illustrates an environment 700 for implementing an implantable or tattooed antenna. In one embodiment, the environment 700 may be a piece of earth. As illustrated, the environment 700 includes a surface 710, a first layer 720 beneath the first layer 710, and a second layer 730 beneath the first layer. It will be appreciated that the environment 700 may include any number of additional layers.

In some embodiments, an implantable antenna 740 may be injected into the first layer 720 by any known technique, such as a technique similar to a tattooing process. The implantable antenna 740 may be made up of nanoparticles in a fluid and thus may operate in a manner similar to that of tattooed radiating antenna 130 previously described. The implantable antenna 740 may be particularly beneficial as a sensing antenna for sensing physical properties of the first layer 740 such as moisture if the first layer is dirt. It will be appreciated by one of skill in the art that implantable antenna 740 may take the form of any reasonable antenna such as those previously described.

FIG. 7 also shows that the implantable antenna 740 may also be injected into second layer 730. This shows that the implantable antenna 740 may also be used in layers that are further from the surface 710.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

We claim:

1. A tattooed antenna comprising:
one or more nanoparticles in a fluid, the one or more nanoparticles being configured to be injected as a tattoo into a body, the one or more nanoparticles forming an antenna configured to transmit data received from a source.

2. The tattooed antenna of claim 1, wherein the one or more nanoparticles are injected subcutaneously.

3. The tattooed antenna of claim 1, wherein the one or more nanoparticles are injected near a surface of the body.

4. The tattooed antenna of claim 1, wherein the one or more nanoparticles are injected into deep tissue that is not near to a surface of the body.

5. The tattooed antenna of claim 1, wherein the antenna is one of a feed pickup antenna or a radiating antenna.

6. The tattooed antenna of claim 1, wherein the antenna comprises a feed pickup antenna and a radiating antenna, the feed pickup antenna receiving the data from the source and providing the data to the radiating antenna and the radiating antenna transmitting the data.

7. The tattooed antenna of claim 1, wherein the source is a feed that is coupled to an implantable medical device that is implanted in the body at a location that is deeper in the body than the location of the antenna.

8. The tattooed antenna of claim 1, wherein the one or more nanoparticle comprise gold.

9. The tattooed antenna of claim 1, wherein the tattooed antenna is injected into a piece of earth.

10. An antenna system comprising:
a feed system coupled to an implantable device that is implanted in a body;
a feed pickup antenna coupled to the feed system so as to be able to receive a data signal from the feed system; and
a radiating antenna coupled to the feed pickup antenna so as to be driven by the feed pickup antenna, the radiating antenna comprising one or more nanoparticles that are tattooed near the surface of a body holding the antenna system.

11. The antenna system of claim 10, wherein the feed pickup antenna is passively coupled to the feed system.

12. The antenna system of claim 10, wherein the feed pickup antenna is directly coupled to the feed system.

13. The antenna system of claim 10, wherein the feed pickup antenna comprises one or more nanoparticles that are tattooed near the surface of a body holding the antenna system.

14. The antenna system of claim 13, wherein the one or more nanoparticles are injected subcutaneously.

15. The antenna system of claim 13, wherein the one or more nanoparticles are injected near a surface of the body.

16. The antenna system of claim 13, wherein the one or more nanoparticles are injected into deep tissue that is not near to a surface of the body.

17. The antenna system of claim 10, wherein the feed system and the implantable device are implanted in the body at a location that is deeper in the body than the location of the radiating antenna.

18. The antenna system of claim 10, wherein the body holding the antenna system is a piece of earth.

19. The antenna system of claim 10, wherein the wherein the one or more nanoparticle comprise gold.

20. The antenna system of claim 13, wherein the wherein the one or more nanoparticle comprise gold.
